SEQUENCE LISTING (7 sequences)

SEQ ID NO: 1 - RNA, 21 nt, Artificial Sequence
  Anti-MVD siRNA
gauggagccuauagaaucauu

SEQ ID NO: 2 - DNA, 22 bp, Artificial Sequence
  sequence is a combined DNA/RNA HMGCoAR 5′ primer
gacgcaacctttatatccgttt

SEQ ID NO: 3 - DNA, 24 bp, Artificial Sequence
  sequence is a combined DNA/RNA HMGCoAR 3′ primer
ttttgaaagtgctttctctgtacc

SEQ ID NO: 4 - DNA, 18 bp, Artificial Sequence
  sequence is a combined DNA/RNA LDLR 5′ primer
gatgtcaatgggggcaac

SEQ ID NO: 5 - DNA, 24 bp, Artificial Sequence
  sequence is a combined DNA/RNA LDLR 3′ primer
tcgttgatgatatctgtccaaaat

SEQ ID NO: 6 - DNA, 20 bp, Artificial Sequence
  sequence is a combined DNA/RNA GAPDH 5′primer
ctctgctcctcctgttcgac

SEQ ID NO: 7 - DNA, 20 bp, Artificial Sequence
  sequence is a combined DNA/RNA GAPDH 3′primer
acgaccaaatccgttgactc

(19) United States
(12) Patent Application Publication
(10) Pub. No.: US 2010/0183613 A1
(43) Pub. Date: Jul. 22, 2010

(54) METHODS OF USING MEVALONATE DECARBOXYLASE (MVD) ANTAGONISTS

(75) Inventors: Jason BORAWSKI, Boston, MA (US); Larry Alexander Gaither, Bradford, MA (US); Mark Aron Labow, Lexington, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG

(21) Appl. No.: 12/582,242

(22) Filed: Oct. 20, 2009

Related U.S. Application Data
(60) Provisional application No. 61/197,003, filed on Oct. 22, 2008.

(51) Int. Cl. A61K 39/395; A61K 31/713; A61K 38/02; A61K 31/357; A61K 31/7088; A61P 31/14

(52) U.S. Cl. 424/135.1; 424/130.1; 514/44 A; 514/2; 514/452; 424/133.1; 424/136.1; 514/44 R

(57) ABSTRACT

The present invention provides novel methods of reducing Flavivirus viral replication and/or infection, e.g., Dengue virus. The invention employs mevalonate decarboxylase (MVD) antagonists to inhibit the cholesterol biosynthesis pathway, thereby inhibiting viral replication/infection.

### BACKGROUND OF THE INVENTION

Dengue is a mosquito borne infection found in tropical and sub-tropical regions around the world. The disease is endemic in more than 100 countries and it is estimated that 250 million people are now at risk of Dengue infection (26, 8). The World Health Organization (WHO) currently estimates that there may be 50 million cases of Dengue infection worldwide every year and global warming provides a significant selective advantage for Dengue infection to continue to spread into new regions. Dengue virus (DENV) is a member of the Flavivirus genus of the Flaviviridae family of enveloped, positive-strand RNA viruses (20) which includes over seventy (70) mosquito-borne and tick borne members, including St Louis encephalitis, Japanese encephalitis, West Nile virus, Dengue 1-4, and yellow fever.

There are four distinct serotypes of Dengue virus DENV 1-4, transmission of which is via mosquitoes, most commonly Aedes aegypti. DENV is unusual among arboviruses in that it does not require a enzootic cycle and is maintained by human-mosquito-human cycle.

The virus is composed of three structural proteins: capsid, C; pre-membrane, prM; envelope, E, and seven viral non-structural proteins: NS1, 2a, 2b, 3, 4a, 4b, and 5 (20). The structure of the virus has been solved by using a combination of cryoelectron microscopy and fitting the known structure of glycoprotein E into the electron density map (17). Binding of the viral E protein to a host receptor DC-SIGN (Dendritic cell (DC)-Specific Intercellular adhesion molecule 3 (ICAM-3) Grabbing Nonintegrin (DC-SIGN), a type II transmembrane protein on DCs with a C-type lectin extracellular domain) is postulated to be one possible route of viral entry to host cells (25).

Dengue virus infections cause a spectrum of diseases, with most cases resulting in asymptomatic infection or Dengue fever (DF), a self limiting illness associated with fever, headache, myalgia (muscle pain), arthralgia (joint pain) and thrombocytopaenia. Infection with one serotype leads to lifelong immunity to that serotype. Re-infection with any of the other three serotypes within 6 months of the first infection leads to protective immunity. After six months the host is susceptible to the other serotypes and a more severe form of the disease known as Dengue Hemorrhagic Fever (DHF) (28). DHF is associated with plasma leakage and bleeding diathesis. DHF patients may progress into Dengue Shock Syndrome (DSS) after 2-7 days of fever, characterized by a rapid, weak pulse, low blood pressure, and in some cases, death.

The pathophysiology of Dengue virus infection is host mediated and not well understood. Secondary infections with a heterologous serotype reportedly lead to more severe disease, suggested to be the result of antibody dependent enhancement (ADE) (9). The ADE model postulates that non-neutralizing antibodies can interact with the DENV and facilitate viral entry via Fc receptors in to monocytes and macrophages (4, 7). The excessive uptake of DENV leads to hyperactivation of T cells and an inappropriate immune reaction. The ADE response induces a proliferation of cytokines in the blood and results in vascular leakage.

Currently there are significant challenges associated with generating a successful vaccine for Dengue virus. Since there are four distinct Dengue serotypes the vaccine would need to generate immunity to each serotype to preclude the development of ADE (31). The ability to generate a robust immunity to four separate genotypes in a single vaccine has yet to be demonstrated. There is a great need for novel targets involved in Dengue virus life cycle, and other Flavivirus viruses, to improve the chances of identifying new approaches for treatment.

### SUMMARY OF THE INVENTION

The present invention provides methods of reducing (e.g., inhibiting) viral infection or viral replication of the Flavivirus family of enveloped, positive-strand RNA viruses in a subject. In a particular embodiment, the methods of the present invention inhibit the infection or replication of the West Nile virus, Japanese encephalitis virus, or Dengue virus, by administration of a therapeutically effective amount of an antagonist of the mevalonate pathway, e.g., a mevalonate decarboxylase (MVD) antagonist.

A wide variety of MVD antagonists can be used in the methods of the present invention, such as small molecules (e.g., statins, CoA synthase inhibitors (e.g., hymeglusin), or squalene synthase inhibitors (e.g., Zaragozic acid A (ZGA)), fusion proteins, nucleic acids (e.g., antisense molecules, such as RNA interfering agents and ribozymes), and MVD-derived peptidic compounds.

In another embodiment, the MVD antagonist is an antibody (of fragment thereof). Antibodies suitable for protection according to the invention include all known forms of antibodies having at least variable region sequences. For example, the antibody can be a murine, human, humanized, chimeric or bispecific monoclonal antibody. The antibody can be a Fab, Fab′2, ScFv, SMIP, affibody, avimer, nanobody, or a domain antibody, and the antibody can be an IgG1, IgG2, IgG3, IgG4, IgM, IgA1, IgA2, IgAsec, IgD, or IgE antibody.

MVD antagonists utilized in the methods of the present invention can be administered alone or in combination with other therapeutic agents. For example, the antibodies can be administered in combination with (i.e., together with or linked to) other known therapeutic agents, i.e., immunosuppressive and/or other therapeutic antibodies. In one embodiment, the antagonist is linked to a second binding molecule, such as an antibody (i.e., thereby forming a bispecific molecule) or other binding agent that binds to a different target or a different epitope on MVD.

Other features and advantages of the invention will be apparent from the following detailed description, and from the claims.

### BRIEF DESCRIPTION OF THE DRAWINGS

FIGS. 1A and 1B show that MVD siRNAs reduce MVD mRNA and suppress Dengue viral replication in A549 replicon cells. (A) A549 Dengue replicon cells were transfected with siRNAs for 72 hours and EnduRen™ Live Cell Substrate (Promega) (the viral reporter renilla) and Cell Titer Glo (CTG) (for cell viability) were analyzed. Each bar represents three independent replicates from two separate experiments. (B) In parallel, the siRNA transfected A549 replicon cells were lysed and mRNA was quantified by RTPCR. Each bar represents three independent mRNA isolates from three siRNA transfections normalized to the internal control actin, and are divided by GAPDH siRNA transfected cells to determine knock clown relative to GAPDH (error bars are present).

FIGS. 2A, 2B, and 2C show that multiple independent siRNAs targeting

MVD reduce Dengue replication and MVD protein levels in A549 cells. (A) A594 replicon cells were transfected with two independent siRNA sequences to MVD, MVD3 and MVD4, for 72 hours and the viral reporter renilla and Cell titer glo luciferase (B) was quantified. Each bar represents three independent replicates from two separate experiments. (C) Protein isolates from siRNA transfected A549 replicon cells were analyzed by Western blot probing both with and anti MVD antibody and actin as the loading control. The Western blot is representative of three independent experiments.

FIGS. 3A-D show that stable knock down of MVD for two weeks suppresses Dengue replicon activity. (A) Short hairpin shRNAs targeting MVD were stably transfected into the A549 replicon cells for two weeks to grow up puromycin resistant pooled clones. Of the five clones, one maintained stable knock down of the MVD protein, MVD5 (D). The renilla levels were reduced ˜4-fold in the MVD5 transduced cells while there was no significant effect on cellular viability as compared to a short hairpin targeting CD3d, a nonspecific control. (B) Each bar represents three independent replicates from the stable cell population. (C) mRNA levels of MVD were quantified on the stable cells to confirm the knock down was maintained over the two week period. Each bar represents three independent mRNA isolates from the stable cells normalized to the internal control actin, and divided by CD3d transduced cells to determine knock clown relative to CD3d (error bars are present).

FIGS. 4A and 4B show that MVD siRNAs reduce Dengue live (NGC strain) virus infection in naïve A549 cells. (A) A549 cells were infected with 1 m.o.i. (multiplicity of infection, i.e., the ratio of infectious agents (e.g., phage or virus) to infection targets (e.g., cell)) of NGC virus for 2 days after the transfection of the validated siRNAs MVD3 and MVD4 as well as the control NS3 siRNA, and virus titer was determined by plaque assay. The infection in no siRNA (Mock) transfected cells was set to 100%. (B) Conditions were the same as for (A), but the cell viability after siRNA transfection and NGC infection was measured by Celltiter Glo luminescence (relative light units, or RLU). Each bar represents three independent replicates from three separate experiments.

FIGS. 5A and 5B show that HMGCoA and LDLR (low density lipoprotein receptor) mRNA levels are reduced after live NGC Dengue infection in A549 cells. (A and B) mRNA levels for HMGCoAR and LDLR were quantified by RTPCR in uninfected A549 cells and A549 cells infected with 1 m.o.i of NGC virus for 24, 48, and 72 hours. Values were normalized to the internal control GAPDH and expressed as relative fold change. Each bar represents three independent wells, infected or uninfected.

FIGS. 6A and 6B show that cholesterol depletion prevents Dengue viral replication over time. Both wild type A549 cells and A549 Dengue replicon cells were grown over a 96 hour time period in complete (filled circles and triangles) or cholesterol delipidated (open circles and triangles) media. (A) Cell titer glo and renilla (B) was quantified every 24 hours. Each point represents replicates of ten from four independent experiments.

FIGS. 7A-C show that the squalene synthesis inhibitor Zaragozic acid A (ZGA) potently inhibits Dengue live virus infection. (A) FACS analysis of K562 cells infected with Dengue NGC and treated with a dose range of ZGA for 48 hours. Cells were labeled with an anti-E antibody 4G2-Alexa 680 and an anti-NS1 antibody labeled with Alexa 688 before FACS. Dengue positive cells (in the upper right corner) constituted 4.8% of the total cell population. After ZGA treatment the percent positive cells dropped from 4.8% to 2.8% at 0.1 μZGA and 0.6% at 10 μM ZGA. (B) The FACS results were confirmed by plaque analysis after 48 hours of ZGA treatment in infected K562 cells. Cells treated with 0.1 μM and 10 μM ZGA formed 2-6 fold less plaque than cells treated with DMSO alone. (C) The ZGA dose treatment had no effect on cell viability as measured by Celltiter Glo. Each FACS plot and bar graph is representative of three independent experiments.

FIGS. 8A-C show that lovastatin inhibits Dengue virus infection in human PBMCs (peripheral blood mononuclear cells). (A) Isolated human PBMCs were infected with Dengue NGC and treated with a dose range of lovastatin for 48 hours. The percentage of 4G2 and NS1 double positive cells were identified by FACS as in FIG. 7 and expressed as a bar graph with increasing concentrations of Lovastatin. (B) The live virus titer in PBMC infection after lovastatin treatment by plaque assay. (C) Cell viability was determined using Celltiter Glo in PBMCs infected with virus and treated with lovastatin for 48 hours. The bar graphs are representative of three independent experiments.

### DETAILED DESCRIPTION OF THE INVENTION

In the Flavivirus family, the host's cholesterol pathway has been shown to be important (19, 21, 23). Although no specific components of the cholesterol pathway have been named as the key regulators, the metabolism of cholesterol is necessary for Flavivirus entry, replication, and the host's immune response to the virus (19, 21, 23).

MVD is an essential enzyme in the cholesterol biosynthesis pathway and has been shown to be a member of the GHMP kinase family with active sites at Asp 302 and Lys 18 (15). MVD is responsible for the production of Isopentenyl 5-Pyrophosphate from mevalonate 5-pyrophosphatate generating CO₂ in the process.

According to the present invention, disruption of host cholesterol biosynthesis through the sterol branch inhibits viral replication, such as, Dengue virus. In particular, pharmacological intervention using statins, hymeglusin, or ZGA can potently inhibit Dengue NGC live virus infection in K562, A549, and human PBMCs, while inhibition of farnesylation or geranylgeranylation (the non-sterol branch of the pathway) has no effect on Dengue.

Accordingly, the present invention relates to methods of reducing (e.g., inhibiting) viral infection or viral replication of the Flavivirus family of enveloped, positive-strand RNA viruses in a subject by disrupting the sterol arm of the cholesterol biosynthesis pathway in a subject. In particular, the invention provides methods of reducing viral infection/replication by disrupting the host mevalonate pathway enzyme mevalonate decarboxylase (MVD) using, for example, a MVD anatagonist. MVD antagonists of the present invention include, for example, small molecules (e.g., statins, CoA synthase inhibitors (e.g., hymeglusin), or squalene synthase inhibitors (e.g., Zaragozic acid A (ZGA)), fusion proteins, nucleic acids (e.g., antisense molecules, such as RNA interfering agents and ribozymes), MVD-derived peptidic compounds, and an antibody (or fragment thereof).

In order that the present invention may be more readily understood, certain terms are first defined. Additional definitions are set forth throughout the detailed description.

### I. DEFINITIONS

As used herein, the term “mevalonate decarboxylase” and “MVD” are used interchangeably and refer to an enzyme in the cholesterol synthesis pathway (GenBank Accession No. NM_{—}002461). MVD is a member of the GHMP kinase family with active sites at Asp 302 and Lys 18 (15). MVD is responsible for the production of isopentenyl 5-pyrophosphate from mevalonate 5-pyrophosphatate, generating CO₂ in the process.

As used herein, and as further defined herein, the term “MVD antagonist” refers to any agent which reduces MVD enzymatic activity relative to its enzymatic activity in the absence of the antagonist, including agents which reduce MVD expression or reduce MVD function (e.g., its ability to produce isopentenyl 5-pyrophosphate from mevalonate 5-pyrophosphatate). Examples of MVD antagonists include molecules, for example, which inhibit nucleic acids that express MVD (e.g., antisense molecules, such as RNA interfering agents and ribozymes), as well as molecules which bind to MVD (e.g., an MVD-antibody or ligand).

As used herein, the term “reduces” refers to any statistically significant decrease in production or biological activity of MVD, including full blocking of production or activity (i.e., inhibition). For example, “reduces” can refer to a decrease of about 10%, 20%, 30%, 40%, 50%, 60%, 70%, 80%, 90%, or 100% in MVD expression, MVD function (e.g., isopentenyl 5-pyrophosphate production), or Flavivirus replication.

As used herein, the term “Flavivirus virus” refers to a virus of the Flaviviridae family of enveloped, positive-strand RNA viruses. Examples of a Flavivirus virus include, but are not limited to, West Nile virus, Japanese encephalitis virus, and Dengue virus. There are four distinct serotypes of Dengue virus DENV1-4, transmission of the virus is via mosquitoes, most commonly Aedes aegypti, and in some cases, tick borne infection. DENV is unusual among arboviruses in that it does not require a enzootic cycle and is maintained by human-mosquito-human cycle. The virus is composed of three structural proteins: capsid, C; pre-membrane, prM; envelope, E, and seven viral non-structural proteins: NS1, 2a, 2b, 3, 4a, 4b, and 5 (20).

### II. MVD ANTAGONISTS

MVD antagonists include any agent which reduces MVD expression, MVD function (e.g., isopentenyl 5-pyrophosphate production), or Flavivirus replication. Representative antagonists include, for example, small molecules (e.g., statins, hymeglusin, or ZGA), antibodies, nucleic acids (e.g., antisense molecules, such as ribozymes and RNA interfering agents), fusion proteins, and MVD-derived peptidic compounds.

A. Small Molecule Inhibitors

In one embodiment, the MVD antagonist employed in the invention is a small molecule inhibitor, such as the small molecule inhibitors used in the Examples described below (e.g., statins, hymeglusin, ZGA). As used herein, the term “small molecule inhibitor” is a term of the art and includes molecules that are less than about 7500, less than about 5000, less than about 1000 molecular weight or less than about 500 molecular weight, and inhibit MVD activity. Exemplary small molecule inhibitors include, but are not limited to, peptides, peptidomimetics, nucleic acids, carbohydrates, small organic molecules (e.g., Cane et al. 1998. Science 282:63), and natural product extract libraries. In another embodiment, the compounds are small, organic non-peptidic compounds. Like antibodies, these small molecule inhibitors can bind to and/or otherwise block MVD-mediated cellular interaction.

B. Antibodies

In another embodiment, the invention employs an antibody that binds MVD and inhibits MVD activity and/or down-modulates MVD expression. For example, the antibody can bind to MVD and interfere with its enzymatic activity. The terms “antibody” or “immunoglobulin,” as used interchangeably herein, include whole antibodies and any antigen binding fragment (i.e., “antigen-binding portion”) or single chains thereof. An “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., MVD). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment (Ward et al. (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242, 423-426; and Huston et al. (1988) Proc. Natl. Acad. Sci. USA 85, 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies can be prepared using any art recognized technique and those described herein such as, for example, a hybridoma method, as described by Kohler et al. (1975) Nature, 256:495, a transgenic animal, as described by, for example, (see e.g., Lonberg, et al. (1994) Nature 368(6474): 856-859), recombinant DNA methods (see, e.g., U.S. Pat. No. 4,816,567), or using phage antibody libraries using the techniques described in, for example, Clackson et al., Nature, 352:624-628 (1991) and Marks et al., J. Mol. Biol., 222:581-597 (1991). Monoclonal antibodies include chimeric antibodies, human antibodies and humanized antibodies and may occur naturally or be recombinantly produced.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric antibody” refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “human antibody,” as used herein, is intended to include antibodies having variable regions in which both the framework and CDR regions are derived from human germline immunoglobulin sequences as described, for example, by Kabat et al. (See Kabat, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The human antibody can have at least one or more amino acids replaced with an amino acid residue, e.g., an activity enhancing amino acid residue which is not encoded by the human germline immunoglobulin sequence. Typically, the human antibody can have up to twenty positions replaced with amino acid residues which are not part of the human germline immunoglobulin sequence. In a particular embodiment, these replacements are within the CDR regions as described in detail below.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79, 315-321; Kostelny et al. (1992) J. Immunol. 148, 1547-1553.

As used herein, a “heterologous antibody” is defined in relation to the transgenic non-human organism or plant producing such an antibody.

An “isolated antibody,” as used herein, is intended to refer to an antibody which is substantially free of other antibodies having different antigenic specificities (e.g., an isolated antibody that specifically binds to MVD is substantially free of antibodies that specifically bind antigens other than MVD). In addition, an isolated antibody is typically substantially free of other cellular material and/or chemicals. In one embodiment of the invention, a combination of “isolated” monoclonal antibodies having different MVD binding specificities are combined in a well defined composition.

As used herein, “isotype” refers to the antibody class (e.g., IgM or IgG1) that is encoded by heavy chain constant region genes. In one embodiment, an antibody or antigen binding portion thereof is of an isotype selected from an IgG1, an IgG2, an IgG3, an IgG4, an IgM, an IgA1, an IgA2, an IgAsec, an IgD, or an IgE antibody isotype.

As used herein, “isotype switching” refers to the phenomenon by which the class, or isotype, of an antibody changes from one Ig class to one of the other Ig classes.

As used herein, “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence regions in a gene encoding an antibody. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibody proteins obtained from members of the camel and dromedary (Camelus bactrianus and Calelus dromaderius) family, including New World members such as llama species (Lama paccos, Lama glama and Lama vicugna), also can be employed in the present invention. Such antibody proteins have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See, for example, PCT Publication WO 94/04678.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain bather. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli.

Accordingly, one type of MVD antagonist that can be employed in the present invention is a camelid antibody or camelid nanobody having high affinity for MVD. In certain embodiments herein, the camelid antibody or nanobody is naturally produced in the camelid animal, i.e., is produced by the camelid following immunization with MVD or a peptide fragment thereof, using techniques described herein for other antibodies. Alternatively, the anti-MVD camelid nanobody is engineered, i.e., produced by selection, for example from a library of phage displaying appropriately mutagenized camelid nanobody proteins using panning procedures with MVD or a MVD epitope described herein as a target. Engineered nanobodies can further be customized by genetic engineering to increase the half life in a recipient subject from 45 minutes to two weeks.

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}-V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21).

A diabody can be fused to Fe to generate a “di-diabody” (see Lu et al., 2004 J. Biol. Chem., 279(4):2856-65).

The invention further includes the use of MVD antagonists that exhibit functional properties of antibodies but derive their framework and antigen binding portions from other polypeptides (e.g., polypeptides other than those encoded by antibody genes or generated by the recombination of antibody genes in vivo). The antigen binding domains (e.g., MVD binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain can be a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones can be created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., hMVD) and for other functions (e.g., inhibition of biological activity of MVD). Selected clones can be used as the basis for further randomization and selection to produce derivatives of higher affinity for the antigen.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci. USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

As used herein, the terms “specific binding,” “specifically binds,” “selective binding,” and “selectively binds,” mean that an antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹M⁻¹, or 10¹⁰ M⁻¹. Affinities greater than 10⁷M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). Specific or selective binding can be determined according to any art-recognized means for determining such binding, including, for example, according to Scatchard analysis and/or competitive binding assays.

The term “K_{D},” as used herein, is intended to refer to the dissociation equilibrium constant of a particular antibody-antigen interaction or the affinity of an antibody for an antigen. In one embodiment, the antibody or antigen binding portion thereof according to the present invention binds an antigen (e.g., MVD) with an affinity (K_{D}) of 50 nM or better (i.e., or less) (e.g., 40 nM or 30 nM or 20 nM or 10 nM or less), as measured using a surface plasmon resonance assay or a cell binding assay. In a particular embodiment, an antibody or antigen binding portion thereof according to the present invention binds MVD with an affinity (K_{D}) of 8 nM or better (e.g., 7 nM, 6 nM, 5 nM, 4 nM, 2 nM, 1.5 nM, 1.4 nM, 1.3 nM, 1 nM or less), as measured by a surface plasmon resonance assay or a cell binding assay. In other embodiments, an antibody or antigen binding portion thereof binds an antigen (e.g., MVD) with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument using recombinant MVD as the analyte and the antibody as the ligand, and binds to the predetermined antigen with an affinity that is at least two-fold greater than its affinity for binding to a non-specific antigen (e.g., BSA, casein) other than the predetermined antigen or a closely-related antigen.

The term “K_{off},” as used herein, is intended to refer to the off rate constant for the dissociation of an antibody from the antibody/antigen complex.

The term “EC50,” as used herein, refers to the concentration of an antibody or an antigen-binding portion thereof, which induces a response, either in an in vitro or an in vivo assay, which is 50% of the maximal response, i.e., halfway between the maximal response and the baseline.

As used herein, “glycosylation pattern” is defined as the pattern of carbohydrate units that are covalently attached to a protein, more specifically to an immunoglobulin protein.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

The term “modifying,” or “modification,” as used herein, is intended to refer to changing one or more amino acids in the antibodies. The change can be produced by adding, substituting or deleting an amino acid at one or more positions. The change can be produced using known techniques, such as PCR mutagenesis. For example, in some embodiments, an antibody employed by the methods of the present invention can be modified, to thereby modify the binding affinity of the antibody to MVD.

The present invention also encompasses “conservative amino acid substitutions” in the sequences of the antibodies used in the methods of the invention, i.e., nucleotide and amino acid sequence modifications which do not abrogate the binding of the antibody encoded by the nucleotide sequence or containing the amino acid sequence, to the antigen, i.e., MVD. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-MVD antibody of the present invention is preferably replaced with another amino acid residue from the same class. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:.412-417 (1997)).

The term “non-conservative amino acid substitution” refers to the substitution of an amino acid in one class with an amino acid from another class; for example, substitution of an Ala, a class II residue, with a class III residue such as Asp, Asn, Glu, or Gln.

Alternatively, in another embodiment, mutations (conservative or non-conservative) can be introduced randomly along all or part of an anti-MVD antibody coding sequence, such as by saturation mutagenesis, and the resulting modified anti-MVD antibodies can be screened for binding activity.

A “consensus sequence” is a sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” of an immunoglobulin refers to a framework region in the consensus immunoglobulin sequence.

Similarly, the consensus sequence for the CDRs of can be derived by optimal alignment of the CDR amino acid sequences of MVD antibodies of the present invention.

C. Nucleic Acids/Antisense Molecules

In another embodiment, the MVD antagonist employed in the present invention is an antisense nucleic acid molecule that is complementary to a gene encoding MVD, or to a portion of said gene, or a recombinant expression vector encoding said antisense nucleic acid molecule. As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

The use of antisense nucleic acids to down-modulate the expression of a particular protein in a cell is well known in the art (see e.g., Weintraub, H. et al., Antisense RNA as a molecular tool for genetic analysis, Reviews—Trends in Genetics, Vol. 1 (1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Antisense nucleic acids can be designed according to the rules of Watson and Crick base pairing. The antisense nucleic acid molecule can be complementary to the entire coding region of MVD mRNA, but more preferably is an oligonucleotide which is antisense to only a portion of the coding or noncoding region of MVD mRNA. For example, the antisense oligonucleotide can be complementary to the region surrounding the translation start site of MVD mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules that can be utilized in the methods of the present invention are typically administered to a subject or generated in situ such that they hybridize with or bind to cellular mRNA and/or genomic DNA encoding MVD to thereby inhibit expression of the MVD, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule employed in the present invention can include an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid used in the present invention is a compound that mediates RNAi. RNA interfering agents include, but are not limited to, nucleic acid molecules including RNA molecules which are homologous to the MVD or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In still another embodiment, an antisense nucleic acid is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988, Nature 334:585-591) can be used to catalytically cleave MVD mRNA transcripts to thereby inhibit translation of MVD mRNA.

Alternatively, gene expression can be inhibited by targeting nucleotide sequences complementary to the regulatory region of MVD (e.g., the MVD promoter and/or enhancers) to form triple helical structures that prevent transcription of the MVD gene in target cells. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12): 807-15.

D. Fusion Proteins and MVD-Derived Peptidic Compounds

In another embodiment, the MVD antagonist used in the present invention is a fusion protein or peptidic compound derived from the MVD amino acid sequence. In particular, the inhibitory compound comprises a fusion protein or a portion of MVD (or a mimetic thereof) that mediates interaction of MVD with a target molecule such that contact of MVD with this fusion protein or peptidic compound competitively inhibits the interaction of MVD with the target molecule. Such fusion proteins and peptidic compounds can be made using standard techniques known in the art. For example, peptidic compounds can be made by chemical synthesis using standard peptide synthesis techniques and then introduced into cells by a variety of means known in the art for introducing peptides into cells (e.g., liposome and the like).

The in vivo half-life of the MVD fusion protein or peptidic compounds of the invention can be improved by making peptide modifications, such as the addition of N-linked glycosylation sites into MVD, or conjugating MVD to poly(ethylene glycol) (PEG; pegylation), e.g., via lysine-monopegylation. Said techniques have proven to be beneficial in prolonging the half-life of therapeutic protein drugs. It is expected that pegylation of the MVD polypeptides of the invention may result in similar pharmaceutical advantages.

In addition, pegylation can be achieved in any part of a polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The MVD polypeptides containing these novel amino acids can then be pegylated at these chosen sites in the protein.

### III. METHODS OF TREATMENT

The present invention provides particular novel therapeutic and diagnostic applications that employ MVD antagonists.

The terms “treat,” “prevent,” “treating,” “preventing,” and “treatment,” as used herein, refer to therapeutic or preventative measures described herein. The methods of “treatment” include administration of a MVD antagonist to a subject in order to prevent, cure, delay, reduce the severity of, or ameliorate one or more symptoms of a disease, condition or infection, in order to prolong the survival of a subject beyond that expected in the absence of such treatment.

The term “patient” includes human and other mammalian subjects that receive either prophylactic or therapeutic treatment.

As used herein, the term “subject” includes any human or non-human animal. For example, the methods and compositions of the present invention can be used to treat a subject having cancer. In a particular embodiment, the subject is a human. The term “non-human animal” includes all vertebrates, e.g., mammals and non-mammals, such as non-human primates, sheep, dog, cow, chickens, amphibians, reptiles, etc.

The term “sample” refers to tissue, body fluid, or a cell from a patient or a subject. Normally, the tissue or cell will be removed from the patient, but in vivo diagnosis is also contemplated. Other patient samples, include urine, tear drops, serum, cerebrospinal fluid, feces, sputum, cell extracts etc.

A. Indications

In one aspect, the methods of the present invention can be used to down-modulate Flavivirus viral replication and/or viral infection in a subject. Exemplary viruses that can be treated and/or diagnosed using the MVD antagonists disclosed herein include, West Nile virus, Japanese encephalitis virus, or Dengue virus.

B. Combination Therapies

MVD antagonists utilized in the methods of the present invention can be administered alone or in combination with other therapeutic agents. For example, the antagonists can be administered in combination with (i.e., together with or linked to (i.e., an immunoconjugate)) other known therapeutic agents (i.e., anti-inflammatory agents, immunosuppressive agents, antiviral agents, and/or other therapeutic antibodies). Examples of anti-inflammatory agents include, for example, aspirin and other salicylates, steroidal drugs, NSAIDs (nonsteroidal anti-inflammatory drugs) (e.g., ibuprofen, fenoprofen, naproxen, sulindac, diclofenac, piroxicam, ketoprofen, diflunisal, nabumetone, etodolac, oxaprozin, and indomethacin), Cox-2 inhibitors e.g., rofecoxib and celecoxib), and DMARDs (disease modifying antirheumatic drugs) (e.g., methotrexate, hydroxychloroquine, sulfasalazine, azathioprine, pyrimidine synthesis inhibitors (e.g., leflunomide), IL-1 receptor blocking agents (e.g., anakinra), TNF-α blocking agents (e.g., etanercept, infliximab and adalimumab), anti-IL-6R antibodies, CTLA4Ig, and anti-IL-15 antibodies). The antagonist can also be administered separate from the agent. In the case of separate administration, the antagonist can be administered before, after or concurrently with the agent or can be co-administered with other known therapies.

In one embodiment, the antagonist is linked to a second binding molecule, such as a second antibody (i.e., thereby forming a bispecific molecule) or other binding agent that binds to a different target or a different epitope on MVD.

C. Dosages/Amounts

The terms “effective amount” and “therapeutically effective amount” as used herein, refers to that amount of an antagonist, which is sufficient to effect treatment, prognosis or diagnosis of an infection or disease associated with increased expression of MVD, as described herein, when administered to a subject. A therapeutically effective amount will vary depending upon the subject and the infection or disease condition being treated, the weight and age of the subject, the severity of the infection or disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody of the present invention. Dosage regimens may be adjusted to provide the optimum therapeutic response. An effective amount is also one in which any toxic or detrimental effects (i.e., side effects) of an antagonist are minimized and/or outweighed by the beneficial effects.

Actual dosage levels of the antagonists used in the methods of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antagonist employed, or the ester, salt or amide thereof, the route of administration, the time of administration, the rate of excretion of the particular antagonist being employed, the duration of the treatment, other drugs, compounds and/or materials used in combination with the particular antagonist employed, the age, sex, weight, condition, general health and prior medical history of the patient being treated, and like factors well known in the medical arts. A physician or veterinarian having ordinary skill in the art can readily determine and prescribe the effective amount of the antagonist required. For example, the physician or veterinarian could start doses of the antagonist at levels lower than that required in order to achieve the desired therapeutic effect and gradually increase the dosage until the desired effect is achieved. In general, a suitable daily dose of an antagonist will be that amount which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of an antagonist may be administered as two, three, four, five, six or more sub-doses administered separately at appropriate intervals throughout the day, optionally, in unit dosage forms. While it is possible for an antagonist of the present invention to be administered alone, it is preferable to administer the antagonist as a pharmaceutical formulation (composition).

Dosage regimens are adjusted to provide the optimum desired response (e.g., a therapeutic response). For example, a single bolus may be administered, several divided doses may be administered over time or the close may be proportionally reduced or increased as indicated by the exigencies of the therapeutic situation. For example, the antagonists used in the methods of the present invention may be administered once or twice weekly by subcutaneous injection or once or twice monthly by subcutaneous injection.

It is especially advantageous to formulate parenteral antagonists in dosage unit form for ease of administration and uniformity of dosage. Dosage unit form as used herein refers to physically discrete units suited as unitary dosages for the subjects to be treated; each unit contains a predetermined quantity of active antagonist calculated to produce the desired therapeutic effect in association with the required pharmaceutical carrier. The specification for the dosage unit forms are dictated by and directly dependent on (a) the unique characteristics of the active antagonist and the particular therapeutic effect to be achieved, and (b) the limitations inherent in the art of compounding such an active antagonist for the treatment of sensitivity in individuals.

D. Methods of Administration and Formulations

To administer an antagonist used in the methods of the present invention by certain routes of administration, it may be necessary co-administer the antagonist with a material to prevent its inactivation. For example, the antagonist may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active antagonist, use thereof in a pharmaceutical compositions is contemplated. Supplementary active compounds can also be incorporated with the antagonist.

Therapeutic antagonists typically must be sterile and stable under the conditions of manufacture and storage. The antagonist can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including an agent that delays absorption, for example, monostearate salts and gelatin.

Sterile injectable solutions can be prepared by incorporating the active antagonist in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Therapeutic antagonists that can be used in the methods of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the antagonist which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed along with the antagonists utilized in the methods of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The antagonists may also be administered with adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the antagonists used in the methods of the present invention are administered to humans and animals, they can be given alone or as a pharmaceutical antagonist containing, for example, 0.001 to 90% (more preferably, 0.005 to 70%, such as 0.01 to 30%) of active ingredient in combination with a pharmaceutically acceptable carrier.

The antagonists can be administered with medical devices known in the art. For example, in a preferred embodiment, an antagonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, antagonists can be formulated to ensure proper distribution in vivo. For example, the blood-brain barrier (BBB) excludes many highly hydrophilic compounds. To ensure that the antagonists cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The present invention is further illustrated by the following examples which should not be construed as further limiting. The contents of Sequence Listing, figures and all references, patents and published patent applications cited throughout this application are expressly incorporated herein by reference.

### EXAMPLES

### Materials and Methods

Materials and Methods

Cells and Viruses

The human lung-epithelial carcinoma A549 cell-line and hematopoetic cell line K562 are from ATCC and cultured according to instruction. PBMC (Peripheral Blood Mononuclear Cell) were prepared from buffy coat from healthy donors using ficoll gradient. The A549 dengue NGC replicon cells used for the work contains non-infectious, self replicating Dengue virus with a Renilla luciferase reporter under puromycin selection (24). Cells were cultured in F12 (Hams) medium (Invitrogen, Carlsbad, Calif.) supplemented with 10% fetal bovine serum (FBS) and puromycin (10-50 μg/ml). Cells were assayed in F12 medium supplement with 2% FBS without puromycin. Dengue NGC virus (GenBank number M29095) and the Malaysian clinical isolates MY10340 (from Shamala Devi, University of Malaya, Malaysia) were propagated in insect cells C6/36 (ATCC). For virus titer analysis, the level of live virus was determined by plaque assay in BHK-21 cells (ATCC) in triplicate.

siRNA Target Validation

Anti-MVD siRNAs were ordered as a Smartpool or individual duplexes (Dharmacon, Lafayette, Colo.). Each siRNA was resuspended in siRNA buffer (Dharmacon, Lafayette, Colo.) to a stock concentration of 20 μM. 2.5 μl of each stock solution was diluted in 197.5 μl of OPTI-MEM 1 (Invitrogen, Carlsbad, Calif.) in a 96 well PCR plate (ABgene # AB-1000) to make a working 10× stamp of 250 μM. For the lipid reagent 0.28 μl of Oligofectamine (Invitrogen, Carlsbad, Calif.) was diluted in 10 μl and added stamped in a 96 well PCR plate (ABgene Rochester, N.Y.). 10 μl of siRNA and 10 μl of lipid was then transferred to each well of a 96 well tissue culture plate (Corning, Corning, N.Y.) and incubated for 20 minutes at room temperature to allow the complexes to form. After the incubation, 6000 A549 cells in 80 μl of assay medium was plated on top of the siRNA:lipid reagent complexes using the Multidrop384 (Thermo, Waltham, Mass.). Cells were assayed as described herein, using EnduRen and CTG. Knock down of mRNA levels was measured using Sybergreen Real-Time PCR. All siRNAs were purchased from Dharmacon ON-TARGETplus collection except for NS3, which was designed in-house, (GAUGGAGCCUAUAGAAUCAUU (SEQ ID NO:1)).

Real-Time PCR

Three wells transfected with siRNAs were pooled together and mRNA was isolated using the TurboCature 8 or 96 kits (Qiagen, Valencia, Calif.). cDNA was generated using a Sprint Powerscript, pre-primed plate (Clontech, Mountain View, Calif.). For the RTPCR, a reaction mix containing 4.2 μl diluted cDNA, 6 μl 2×PCR buffer (Applied Biosystems, Foster City, Calif.), 0.6 μL GOI probe labeled with FAM/MGH and 0.6 μl of β-Actin probe labeled with VIC/TAMRA was set up in 384 well plates. The mRNA levels were measured using Syber green on an Applied Biosystems 7900HT (Applied Biosystems, Foster City, Calif.) using gene specific primers ordered from Applied Biosystems.

For HMGCoAR and LDLR real-time PCR, total RNA were extracted using Rneasy kit (Qiagen). 20 ng of total RNA was used in the iScript One-step RT-PCR Sybro Green kit (Biorad) using HMGCoAR, LDLR and GAPDH specific 5′ and 3′ primers (HMGCoAR 5′ primer: GACGCAACCTTTATATCCGTTT (SEQ ID NO:2), 3′ primer: TTTTGAAAGTGCTTTCTCTGTACC (SEQ ID NO:3); LDLR 5′ primer: GATGTCAATGGGGGCAAC (SEQ ID NO:4), 3′ primer: TCGTTGATGATATCTGTCCAAAAT (SEQ ID NO:5); GAPDH 5′ primer: CTCTGCTCCTCCTGTTCGAC (SEQ ID NO:6), GAPDH 3′ primer: ACGACCAAATCCGTTGACTC (SEQ ID NO:7)) according to the Biorad protocol. The 15 ul reaction was loaded in 96 well plate and run with a Biorad iQ5 machine.

EnduRen Live Cell Substrate Assay

A549 cells were cultured for a total of 72 h in assay medium. Cells were pulsed for the final 2 h with 60 μM EnduRen. Luminescence with EnduRen reaches a maximum after 1.5 h but remains stable for greater than 24 h (Promega, Madison, Wis.). Luminescence was measured using an EnVision™ 2100 multilabel reader (PerkinElmer, Waltham, Mass.). 60 mM stocks of EnduRen (Promega, Madison, Wis.) were prepared in DMSO and stored at −20° C. Stocks were diluted 1:100 into pre-warmed assay medium. Subsequent 1:10 dilutions were made by adding 10 μl of diluted EnduRen to each well of a 96-well plate containing 100 μL of cells, for a final concentration of 60 μM. 2.5 CellTiter-Glo (CTG) Luminescent Cell Viability Assay (Promega, Madison, Wis.). The assay was used according to the manufacture's instructions. Substrate and buffer were combined and mixed to form the assay reagent. Reagent was either used immediately or aliquoted and stored at −20° C. An equal volume of reagent to final assay volume is added to the assay plate following determination of Renilla luciferase levels. CTG luminescence was read after 10 minutes. Luminescence was measured using an EnVision 2100 multi-label reader (PerkinElmer, Waltham, Mass.). The same assay plate was used for both EnduRen and CTG reads due to the difference in signal strength.

Low Molecular Weight Inhibitors

Statins were run in A549 Dengue replicon assays by plating out in assay medium into costar 96 well white plates (Corning, Corning, N.Y.) at 6,000 cells per well and left overnight in a humidified 37° C. incubator. The following day two-fold serial dilutions of the compounds were prepared in assay medium and stamped out in a 96 well PCR plate (ABgene Rochester, N.Y.). 10 μl of 10× compound was then added to the plates using the Biomek FX robot. A 12 point dose curve starting at 50 μM was run and after 72 h compound addition, plates were assayed with EnduRen and CTG, to measure virus and cell viability respectively. Samples were singlets, but two identical plates were run and values averaged. All of the Statins and Zargozic acid A (ZGA) stocks were purchased from Sigma.

Western Blot Analysis

For western blot analysis, cells were cultured in 6 well plates at 200,000 cells per well and treated as described herein. Briefly 200 μl lipid and 200 μl siRNA were preincubated for 20 min on the plates. Cells were added to the plate in 1 ml of assay medium, a further 400 μl of medium was added and the cells incubated overnight at 370 C. The following day, 200 μl of 10× compound was added in 200 μl assay medium. After a further 72 h, the medium was removed and wells were washed twice with ice cold PBS. 300 μl of cold lysis buffer, 25 ml RIPA (Boston Bioproducts, Worcester, Mass.) plus protease inhibitor tablet (Roche, Basel, Switzerland), was then added to each well for 20 min on ice with rocking. Cell lysates were transferred to Eppendorf tubes and centrifuged at maximum speed for 15 min at 4° C. to remove cell debris. Protein concentration was determined by BCA assay (Pierce, Rockford, Ill.).

Briefly, in a 96 well UV plate (Corning, Corning, N.Y.) 2 μl of cell lysate was added to 18 μl of PBS then 200 ul of per-prepared protein assay solution was added and concentration read Versamax (Molecular Devices, Sunnyvale, Calif.). SDS-PAGE and protein transfer for western blot analysis 8-10 μg per lane of total protein was loaded. Prior to boiling lysates for 4 min, 7 μl of 6× laemli buffer was added and the samples made up to 45 μl. Boiled, cooled, centrifuged samples were loaded onto a 4-12% tris-glycine pre-cast gel (Invitrogen, Carlsbad, Calif.). Proteins were separated using 1× tris-glycine SDS running buffer (Bio-Rad, Hercules, Calif.) at 120V for 1.5 h. Proteins were transferred to nitrocellulose using the tank transfer method in 1× tris-glycine transfer buffer (Bio-Rad, Hercules, Calif.) at 100 V for 1.5 h at 4° C.

Antibody incubation

Post transfer, the membrane was removed from the transfer tank and incubated with Ponceau S solution (Sigma, St. Louis, Mo.) for 2 min to visualize total protein transfer. The blots were then rinsed with water to visualize individual bands, then washed with 0.1 M NaOH to remove the stain. The blots were blocked in blocking buffer (1×PBS, 5% w/v blotting grade blocker nonfat dry milk (Bio-Rad, Hercules, Calif.) overnight 4° C. with gentle rocking. The membranes were probed with either 1 ug/ml anti-MVD clone 2A7 (Affinity BioReagents, Golden, Colo.), or 1:5000 anti-actin clone AC-15 (Sigma, St. Louis, Mo.). An anti-mouse HRP secondary antibody was obtained from Sigma. All incubations were in blocking buffer for 1 h at room temp followed by three washes. All dilutions and washes were with gentle rocking. SuperSignal West Pico Chemiluminescent Substrate kit (Pierce, Rockford, Ill.) and Kodak's X-Omat AR-2 Film were used to develop the signal.

Infectious Virus Experiments, FACS Measurement and EC50/CC50 Calculation

For siRNA testing, the same individual MVD duplexes were transfected (10 pM) with 0.15 ul of Lipofectamine™ RNAi Max (Invitrogen) in 20 μL of OPTI-MEM into 10⁵ A549 cells per well of a 96 well plate and one day after, NGC virus was added at 1 m.o.i for two days. The virus titer was determined by plaque assay (Morens D M, et al. (1985) J Clin Microbiol. 22(2):250). The cell viability was determined by adding 100 μl of Celltiter Glo reagent (Promega) and read with a luminescence plate reader. For compound testing, K562 infection system was used. 50,000 K562 cells were plated in 96 well in 100 ul and infected the second day with 1 m.o.i of NGC virus for two days. Compounds were added together with the virus and the viral growth was measured by FACS with two antibodies, the anti-Dengue Envelope protein antibody (ATCC) (26) conjugated to Alexa 680 (Invitrogen) and the anti NS1 antibody (ATCC) conjugated to Alexa 488. The cell viability is measured by Celltiter Glo. The supernatant after infection were collected for plaque assay for live virus titer. For PBMC infection, conditions were the same as for K562 infection except 500,000 total cells were plated and an 1:10,000 diluted dengue IgG positive serum was added to the infection.

To determine the EC50 (effective concentration of 50% inhibition of virus growth) and the cytotoxity CC50 (concentration of 50% cell death), a dose response curve covering at least four different concentrations was done in K562 infection. The decrease of the percentage of the dengue positive cells was then plotted against the concentrations of inhibitors, using a nonlinear regression analysis to obtain the EC50. CC50 was calculated using the same method using the cell viability over the same range of inhibitor concentrations.

### Example 1

### siRNA Analysis of Cholesterol Biosynthetic Genes

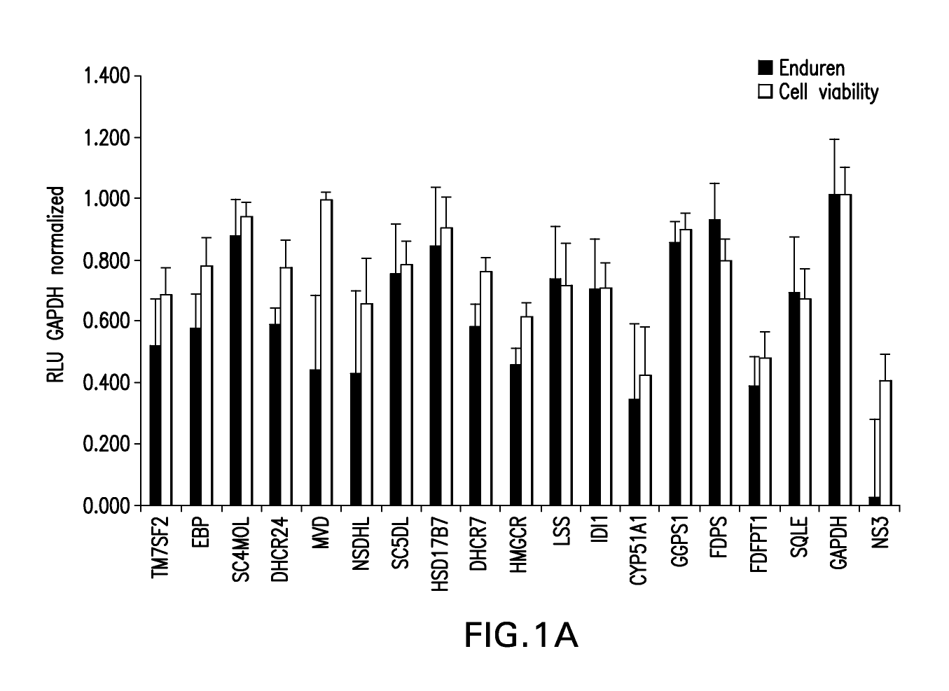

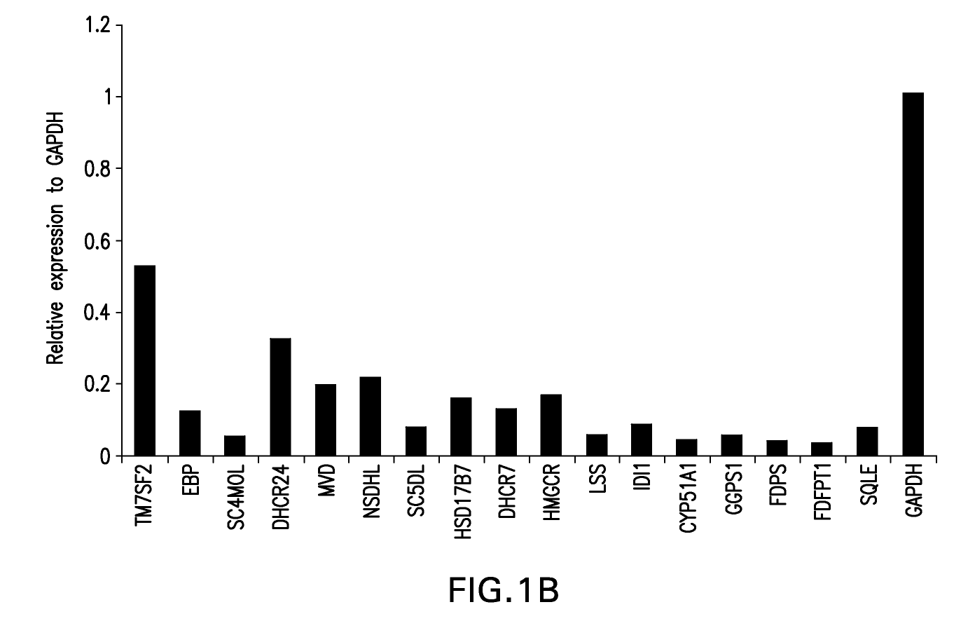

To determine which genes in the cholesterol pathway play a critical role in the replication of the Dengue virus (Flavivirus), a candidate gene-by-gene approach was employed to target cholesterol metabolism using siRNA knock down. Genes with high expression were selected in A549 cells and were either essential or branch point enzymes in the cholesterol biosynthesis pathway (Table 1). Each siRNA was transfected into the A549 Dengue replicon cells as smart pools using siRNA transfection optimization procedures (3). After 72 hours of transfection, cell viability and EnduRen™ levels were measured along with RTPCR (FIGS. 1A and B). The siRNA smart pool targeting MVD (mevalonate diphospho decarboxylase) knocked down target mRNA levels by 80% and had the most significant effect of EnduRen™ levels, 50%. Although most of the siRNAs could potently knock down their respective mRNAs, the phenotypes were modest.

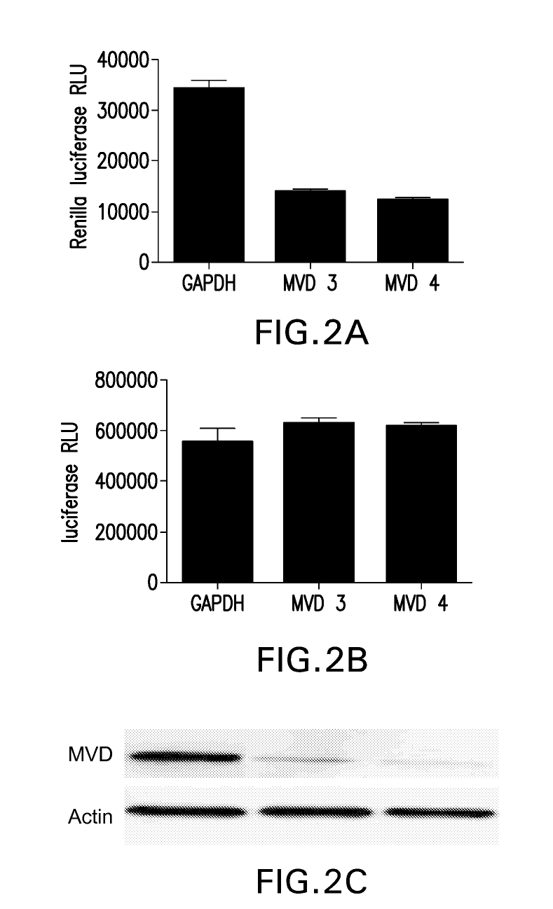

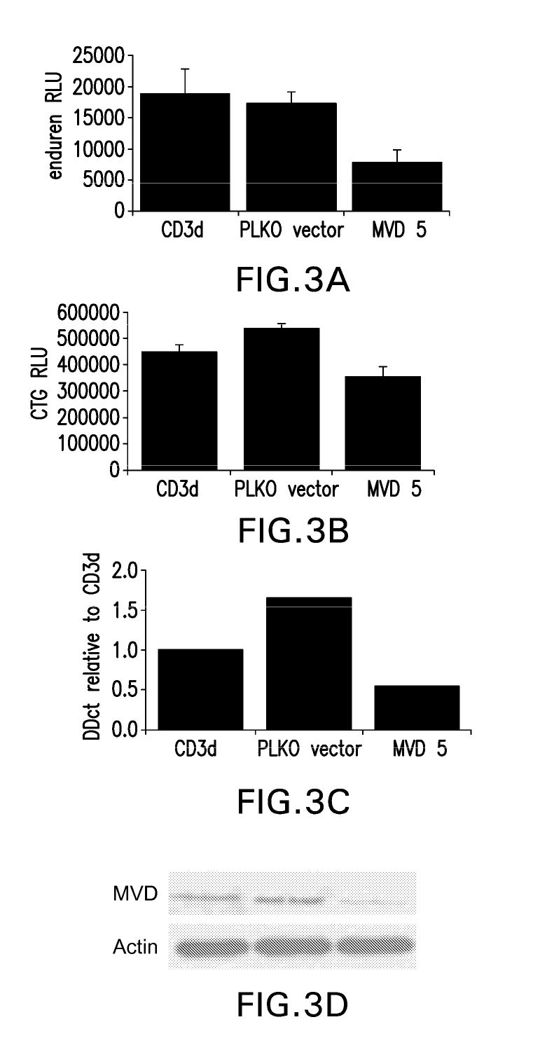

To further validate the role of MVD in viral replication, multiple independent siRNAs targeting MVD in the replicon cells were analyzed. Of the four siRNAs analyzed, two siRNAs could inhibit replicon activity ˜3-fold with no effect on cell viability (FIGS. 2A and B). Both siRNAs could knock down not only mRNA levels >80% (data not shown) but significantly inhibited protein levels (FIG. 2C). shRNAs were also designed to MVD to examine the effects of long term target knock down in the replicon cells. Of the five shRNAs used to establish a stable cell, one construct was found to be active (FIG. 3). The active construct could inhibit replicon activity ˜3-fold over the two week assay, and protein levels were also knocked down over this time period (FIGS. 3A and C). The loss of MVD in the replicon cells was not overtly toxic to the cells after two weeks of knock down (FIG. 3B).

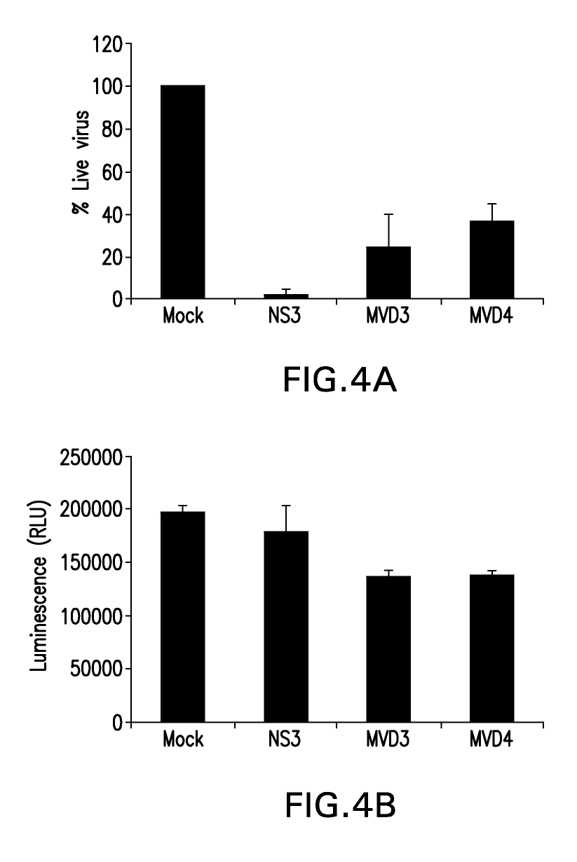

Once the siRNAs were validated in the replicon cells they were analyzed in the Dengue live virus infectious assay. The live Dengue virus was added 24 hours after the siRNA transfection along with validated siRNAs targeting the NS3 protease in the Dengue genome. Both MVD siRNAs could inhibit Dengue infection ˜2-fold compared with the mock transfected control with no significant effect on cell viability (FIGS. 4A and B). The reduction of infectious virus established that loss of MVD was critical not only in the replicon context but also in the infection setting.

TABLE 1
Cholesterol biosynthetic genes targeted with siRNAs

| Gene Name | Gene Function | RefSeqID |
|---|---|---|
| HMGCR | 3-hydroxy-3-methylglutaryl-Coenzyme A reductase | NM_000859 |
| MVD | mevalonate (diphospho) decarboxylase | NM_002461 |
| IDI1 | isopentenyl-diphosphate delta isomerase 1 | NM_001003686 |
| GGPS1 | geranylgeranyl diphosphate synthase 1 | NM_001037277 |
| FDPS | farnesyl diphosphate synthase | NM_002004 |
| FDFPT1 | farnesyl-diphosphate farnesyltransferase 1 | NM_004462 |
| SQLE | squalene epoxidase | NM_003129 |
| LSS | lanosterol synthase | NM_001001438 |
| CYP51A1 | cytochrome P450, family 51, subfamily A, polypeptide 1 | NM_000786 |
| TM7SF2 | transmembrane 7 superfamily member 2 | NM_003273 |
| SC4MOL | sterol-C4-methyl oxidase-like | NM_001017369 |
| NSDHL | NAD(P) dependent steroid dehydrogenase-like | NM_015922 |
| HSD17B7 | hydroxysteroid (17-beta) dehydrogenase 7 | NM_016371 |
| EBP | emopamil binding protein (sterol isomerase) | NM_006579 |
| DHCR24 | 24-dehydrocholesterol reductase | NM_014762 |
| SC5DL | sterol-C5-desaturase | NM_001024956 |
| DHCR7 | 7-dehydrocholesterol reductase | NM_001360 |

### Example 2

### Host Cholesterol Pathway Changes During Infection

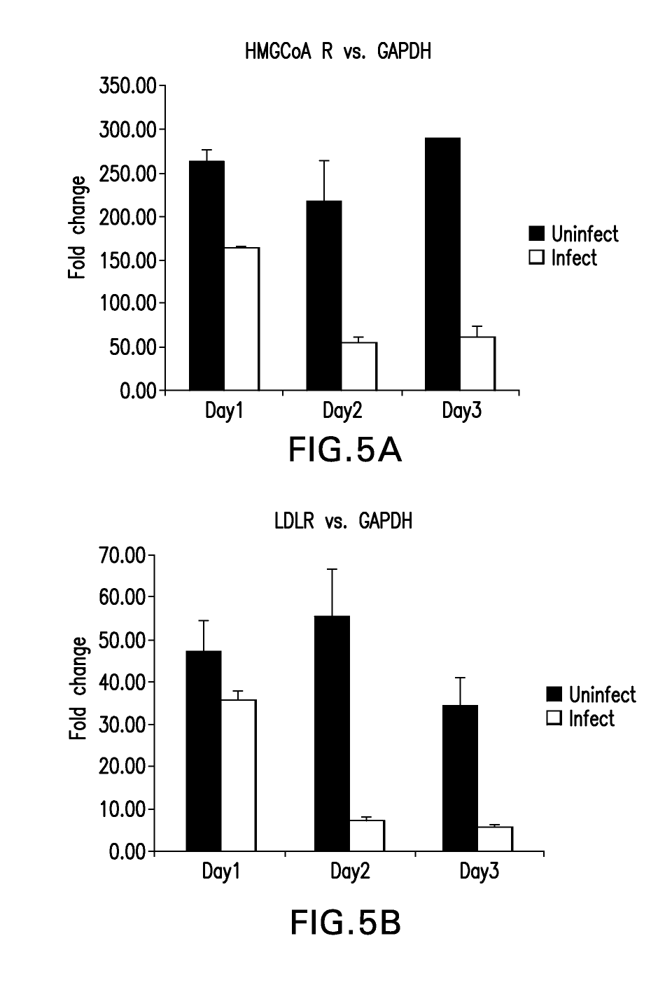

Targeting MVD inhibits endogenous cholesterol production, as well as increases mevalonic acid levels in the cell. It is possible Dengue replication depends on the production of endogenous cholesterol production because the virus requires lipid rafts to replicate and traffic in the cell (19). To explore how cholesterol levels are affected by Dengue infection, the cholesterol content in A549 cells were measured after Dengue live virus infection for various times. The total cholesterol content of the cells did not change significantly after infection. Cholesterol is most enriched in the plasma membrane and is less abundant in the endoplasmic reticulum (13). Although the total cholesterol did not change, a local change of cholesterol at the replication site cannot be ruled out. To detect the potential local change in cholesterol, the messenger level of HMGCoAR and LDLR (whose transcriptions were tightly controlled by the level of cholesterol in the endoplasmic reticulum, where the dengue replication takes place) were measured. As shown in FIGS. 5A and 5B, after normalizing the expression level with a housekeeping gene GAPDH, the transcript level of HMGCoAR and LDLR stayed unchanged in the uninfected cells for 3 days while the level of both genes decreased significantly starting from day 2 post infection. The cholesterol biosynthesis pathway genes are among the most sensitive genes for feedback transcriptional control. The suppressed HMGCoAR and LDLR gene transcription indicated that there is an elevated cholesterol content in the endoplasmic reticulum.

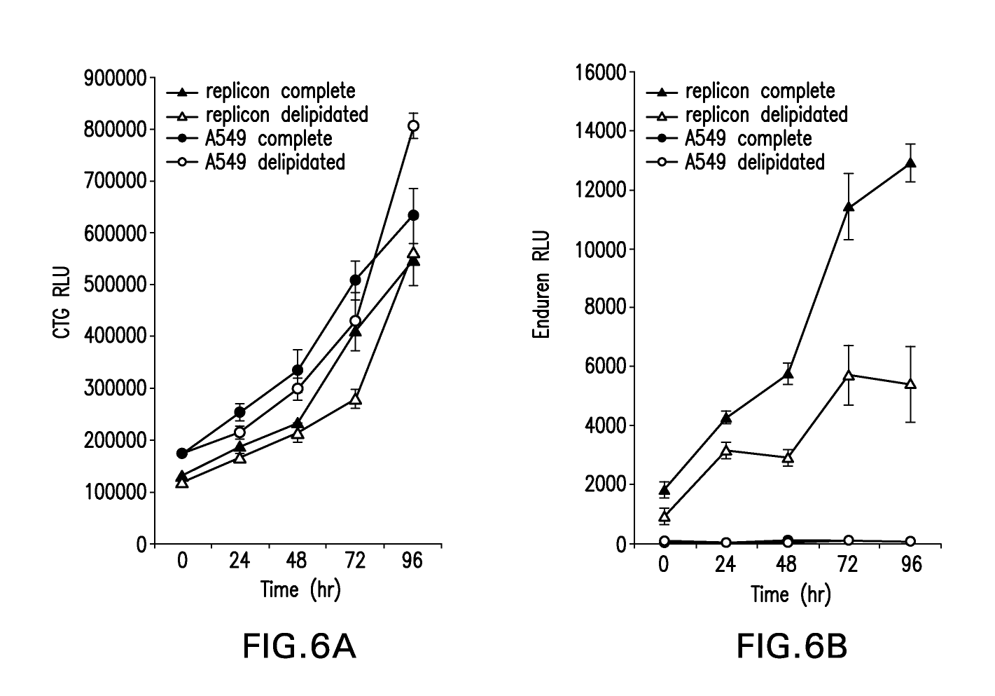

To further elucidate the need of cholesterol for Dengue replication, replicon cells were grown in cholesterol depleted media over a four day time period measuring both cell viability replicon activity. The cells underwent two doublings in four days and grew at the same rate in complete or delipidated media in both wild type and Dengue replicon cells (FIG. 6A). Although the replication activity correlated with growth in complete media, the cells in delipidated media had only 2-fold the level of EnduRen™. Thus, cholesterol levels play an active role in maintaining replicon activity in the stable subgenomic cell line where virus replication is established.

### Example 3

### Pharmacological Inhibition of Cholesterol Biosynthesis

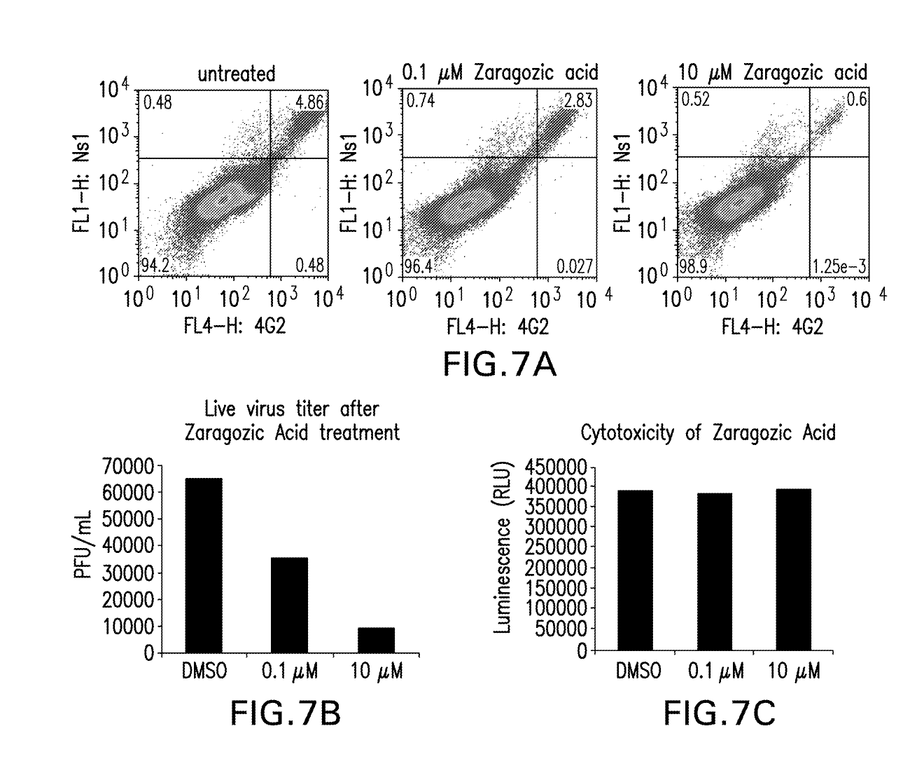

To determine if endogenous cholesterol production was important for Dengue replication, several known cholesterol inhibitors were analyzed in virus infection in K562 cells (a hematopoetic cell line derived from human chronic myelogenous leukaemia cells). As shown in FIG. 7A, about 5% of K562 were infected by 1 m.o.i of NGC strain of virus after two clays, while the treatment with increasing concentrations of the Squalene synthase inhibitor ZGA decreased the percentage of Envelop and NS1 double positive cell population to only 0.6% at 10 μM. The inhibition of ZGA on infection could be confirmed by plaque assay of the supernatant after infection (FIG. 7B), while no significant cytotoxicity of the inhibitor could be observed (FIG. 7C). Using the same method, other inhibitors of the cholesterol biosynthesis pathway were tested and established the EC50 (effective concentration of 50% inhibition of virus growth) and CC50 (cytotoxity of 50% cell death) of these inhibitors. Table 2 is the summary of these inhibitors in the K562 infection assay highlighting the EC₅₀ and CC₅₀ values. Only the HMG CoA synthase inhibitor Hymeglusin and squalene synthase inhibitor ZGA significantly inhibited viral growth. The therapeutic index (TI) for inhibition of the virus over cell viability was 12 and 6-fold for Hymeglusin and ZGA, respectively. In contrast, the genranylgeranyl transferase and farnesyl transferase inhibitors had no effect on the infection. Several HMGCoA reductase inhibitors (Statins) were also tested and found they caused significant cytotoxicity in K562 cells, which prevented their assessment regarding antiviral effect.

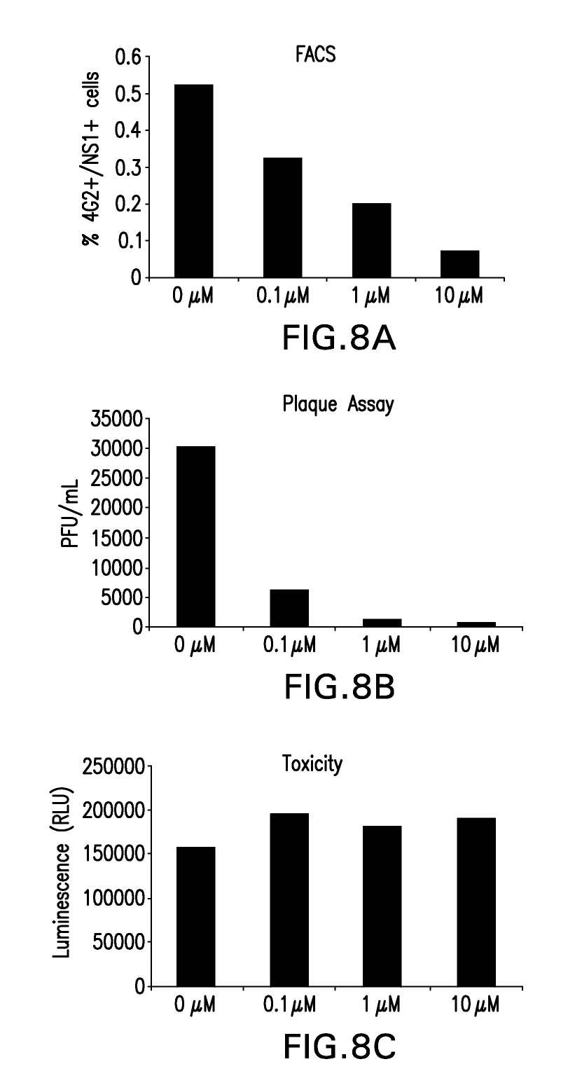

Statins are a safe and widely used drug for the treatment of hypercholesterolemia (12). The toxicity observed in K562 cells could be due to the specific effect of Statins in a cancer cell line (1,22). For avoiding the toxicity in cancer or transformed cell lines, health donors' PBMC were collected, and tested the effect of statin in PBMC infection was tested. PBMCs were infected with a clinically isolated dengue strain MY10340 for two days in the presence of enhancing antibodies from dengue positive plasma. The infection was assessed by FACS and plaque assay as for K562 infection. As shown in FIG. 8, both the percentage of infected cells and the virus titer decreased with increasing concentration of Lovastatin while cytotoxicity was not observed. Overall, pharmacological inhibitors of HMGCoA synthase, HMGCoA reductase and squalene synthase all showed clear inhibition of infection while genranylgeranyl transferase and farnesyl transferase inhibitors had no effect, pointing to the cholesterol biosynthesis pathway, especially the sterol branch of the pathway is involved in dengue virus infection.

TABLE 2
Pharmacological inhibitors of Dengue
live virus infection in K562 cells

| Class | Compound | EC50 | CC50 |
|---|---|---|---|
| Synthetic Statin | Fluvastatin | >50 μM | 48.8 μM |
| Natural Statin | Lovastatin | >50 μM | 46.8 μM |
| HMGCoA synthase inhibitor | Hymeglusin | 4.5 μM | ~50 μM |
| Squalene Synthase inhibitor | Zaragozic Acid | 8.3 μM | >50 μM |
| Farnesyl transferase inhibitor | FTI III | >50 μM | >50 μM |
| Geranylegeranyl transferase | GGTI 2133 | >50 μM | >50 μM |
| inhibitor | | | |

Using a combination of RNAi knock down techniques and pharmacological inhibitors, it was found that sustained endogenous production and exogenous cholesterol uptake are essential for Dengue virus replication and uptake. After screening a collection of siRNAs targeting key nodes of the cholesterol biosynthetic pathway, MVD was identified as essential for viral replication when tested in the stable replicon cells (FIGS. 1 and 2). Stable knock down of MVD using a short hairpin shRNA for two weeks confirmed that MVD can be reduced without killing the cells while virus replication is compromised (FIG. 3). The siRNA and shRNA knock clown experiments show MVD knock down can inhibit the subgenomic replicon of Dengue. When the validated siRNAs targeting MVD are transfected prior to live Dengue infection, viral infection is also significantly inhibited (FIG. 4). This data shows that, in addition to inhibiting established replication in the stable Dengue replicon cells, live virus infection can also be prevented. The RNAi data collectively demonstrates disruption of the mevalonate pathway through the host enzyme MVD is necessary for Dengue virus replication and infection in A549 cells.

MVD catalyzes the decarboxylation of mevalonic acid pyrophosphate to form isopentenylpyrophosphate, a key intermediate in the cholesterol cascade. Although MVD is not the rate limiting reaction, the phosphorylated intermediates serve regulatory functions in lipid biosynthesis including a reduction of cholesterol from acetate. In addition, the inhibition of MVD leads to an increase in mevalonic acid pyrophosphate linked to the regulation of fatty acid synthetic enzymes (16). There are known genetic mutations in the MVD pathway associated with loss of the kinase activity of the MVD phosphorylating enzyme MVK (5). Thus, the inhibition of MVD in the Dengue replicon cells results in not only a reduction in cellular cholesterol levels, but the accumulation of phosphorylated intermediates such as Phospho- and pyrophospho-mevalonic acid.

As described above, to understand the requirement for MVD in dengue replication, it was tested whether cholesterol levels in dengue infected cells were changed. Although no significant change in the total cholesterol content of the cell was found, the messenger level of HMGCoAR and LDLR was measured. Under conditions of low cholesterol in the endoplasmic reticulum, the cholesterol sensor SREBP (Sterol regulatory element binding protein)-SCAP (SREBP cleavage activating protein) complex is transported to the Golgi compartment where S1P/S2P proteases can cleave the cytoplasmic domain of SREBP to allow it to translocate into the nucleus and act as a transcription factor to induce HMGCoAR and LDLR gene transcription (30). Since it was observed that infection suppressed the transcript level of both HMGCoAR and LDLR, it indirectly indicates that ample (if not excess) cholesterol was present in the endoplasmic reticulum and this was caused by dengue replication that occurs on the membrane of the endoplasmic reticulum. It has been reported that West Nile infection caused a relocalization of plasma membrane cholesterol to the replication site and that HMGCoAR was found to be colocalizing with viral protein (21). In dengue infection, it was reported recently that the Dengue replication complex is located in the cholesterol rich micro domains in the endoplasmic reticulum (19). These data, together with our findings, demonstrate that Dengue, as well as West Nile virus, require cholesterol mobilization to maintain replication in cells.

To confirm that exogenous cholesterol was essential for Dengue replication, stable replicon cells were grown in cholesterol depleted media for 4 days (FIG. 6). Although all the cells grew normally when cholesterol was depleted, the replication of the virus was inhibited by 2-fold. Since the cells were “shocked” by transfer from complete to depleted media that the endogenous stores of cholesterol would be replete. It was surprising that 96 hours could still slow clown stable viral replication, suggesting the demand for exogenous cholesterol is significant. This is consistent with the findings of Lee et al. that exogenous cholesterol chelation could reduce intracellular replication of JEV and DEN-2 (19).

As described above, pharmacological tool compounds were used to validate the hypothesis that Dengue replication requires cholesterol production. There are a number of cholesterol biosynthetic inhibitors including statins, squalene synthesis inhibitors, geranylgeranylation inhibitors, and farnesylation inhibitors all of which can inhibit cholesterol production or the non-sterol branch of the pathway. Interestingly, the statins and HMGCoA synthase inhibitor hymeglusin, squalene synthesis inhibitor zaragozic acid could potently inhibit Dengue infection (Table 2, FIGS. 7 and 8) while the geranylgeranylation inhibitors and farnesylation inhibitors had no effect. The sensitivity of K562 cells to statins is not surprising as rapidly growing cancer cells are sensitive to HMGCoA inhibition (1). The results of statins' blocking live virus replication in PBMC provides evidence that molecules that have been shown to be safe in man could be used to block Dengue virus.

Cholesterol pools are dynamic and in a constant state of flux between membranes and biosynthetic pathways. The data shows that protein farnesylation and geranylation have no obvious effects on Dengue replication (Table 2). Although most of the data supports cholesterol as the essential biomolecule for Dengue replication, other downstream metabolites of cholesterol, such as ecosanoids or leukotriens, have not yet been ruled out. Cholesterol is known to increase inflammatory responses and statins can suppress cholesterol induced inflammation. Not only could cholesterol lowering agents help prevent Dengue replication, but might prevent hemorrhagic cases of Dengue fever as statins are also anti-inflammatory (18).

The Dengue virus requires cholesterol to maintain its replication life cycle in cells (FIG. 9). The use of stains to block infectious virus provides an important opportunity to safely treat Dengue fever. The genetic evidence that reduction of MVD can inhibit both a subgenomic replicon and live Dengue virus shows another target in the cholesterol biosynthesis pathway for which small molecules could be developed. The use of squalene synthetase inhibitors (i.e.-ZGA) has already been demonstrated to be efficacious using in vivo animal models (2). The results demonstrate the pharmacological intervention of the cholesterol pathway is a viable therapeutic intervention point to treat Dengue fever.

### INCORPORATION BY REFERENCE

All publications, patents, and pending patent applications referred to herein are hereby incorporated by reference in their entirety.

Sequence Listing

| SEQ ID NO | Sequence |
|---|---|
| SEQ ID NO: 1 | GAUGGAGCCUAUAGAAUCAUU |
| SEQ ID NO: 2 | GACGCAACCTTTATATCCGTTT |
| SEQ ID NO: 3 | TTTTGAAAGTGCTTTCTCTGTACC |
| SEQ ID NO: 4 | GATGTCAATGGGGGCAAC |
| SEQ ID NO: 5 | TCGTTGATGATATCTGTCCAAAAT |
| SEQ ID NO: 6 | CTCTGCTCCTCCTGTTCGAC |
| SEQ ID NO: 7 | ACGACCAAATCCGTTGACTC |

### REFERENCES CITED

1. Bennis, F., Favre, G., Le Gaillard, F., and Soula, G. 1993. Importance of mevalonate-derived products in the control of HMG-CoA reductase activity and growth of human lung adenocarcinoma cell line A549. Int J. Cancer. 55(4):640-5.
2. Bergstrom, J. D., Kurtz, M. M., Rew, D. J., Amend, A. M., Karkas, J. D., Bostedor, R. G., Bansal, V. S., Dufresne, C., VanMiddlesworth, F. L., Hensens, O. D., Liesch, M., Zink, D. L., Wilson, K. E., Onishi, J., Milligan, J. A., Bill, G., Kaplan, L., Nallin Omstead, M., Jenkins, R. G., Huang, L., Meinz, M. S., Quinn, L., Burg, R. W., Kong, Y. L., Mochales, S., Mojena, M., Martin, I., Pelaez, F., Diez, M. T., and Alberts, A. W. 1993. Zaragozic acids: a family of fungal metabolites that are picomolar competitive inhibitors of squalene synthase. Proc Natl Acad Sci USA. 90(1): 80-4
3. Borawski, J., Lindeman, A., Buxton, F., Labow, M., and Gaither, L. A. 2007. Optimization procedure for small interfering RNA transfection in a 384-well format. J Biomol Screen. 12(4):546-59.
4. Clyde, K., Kyle, J. L., and Harris, E. 2006. Recent advances in deciphering viral and host determinants of dengue virus replication and pathogenesis. J. Virol. 80(23): 11418-31.
5. Cuisset, L., Drenth, J. P., Simon, A., Vincent, M. F., van der Velde Visser, S., van der Meer, J. W., Grateau, G. and Delpech, M. 2001. Molecular analysis of MVK mutations and enzymatic activity in hyper-IgD and periodic fever syndrome. Eur J Hum Genet. 9:260-6.
6. del Real, G., Jiménez-Baranda, S., Mira, E., Lacalle, R. A., Lucas, P., Gómez-Mouton, C., Alegret, M., Peña, J. M., Rodríguez-Zapata, M., Alvarez-Mon, M., Martinez-A. C., and Mañes, S. 2004. Statins inhibit HIV-1 infection by down-regulating Rho activity. J Exp Med. 200(4):541-7.
7. Green, S, and Rothman, A. 2006 Immunopathological mechanisms in dengue and dengue hemorrhagic fever. Curr Opin Infect Dis. 19(5):429-36.
8. Gubler, D. J. 1997. Dengue and dengue hemorrhagic fever: its history and resurgence as a global public health problem. Dengue and Dengue Hemorrhagic Fever. CAB International, Wallingford, UK, 1-22.
9. Halstead, S. B., Nimmannitya, S., and Cohen, S. N. 1970. Observations related to pathogenesis of dengue hemorrhagic fever. IV. Relation of disease severity to antibody response and virus recovered. Yale J Biol Med. 42:311-328.
10. Fink J, Gu F, Ling L, Tolfvenstam T, Olfat F, Chin K C, Aw P, George J, Kuznetsov V A, Schreiber M, Vasudevan S G, Hibberd M L. 2007. Host gene expression profiling of dengue virus infection in cell lines and patients. PLoS Negl Trop Dis. 1(2):e86.
11. Ikeda, M., Abe, K., Yamada, M., Dansako, H., Naka, K., and Kato, N. 2006 Different anti-HCV profiles of statins and their potential for combination therapy with interferon. Hepatology. 44(1):117-25.
12. Itakura, H., Kita, T., Mabuchi, H., Matsuzaki, M. Matsuzawa, Y., Nakaya, N., Oikawa, S, Saito, Y., Sasaki, J., and Shimamoto, K. 2008. The J-LIT Study Group Relationship Between Coronary Events and Serum Cholesterol During 10 Years of Low-Dose Simvastatin Therapy. Circ J. 72(8):1218-1224.
13. Jingami, H., Brown, M S., Goldstein, J. L., Anderson, R. G., and Luskey, K. L. 1987. Partial deletion of membrane-bound domain of 3-hydroxy-3-methylglutaryl coenzyme A reductase eliminates sterol-enhanced degradation and prevents formation of crystalloid endoplasmic reticulum. J. Cell Biol. 104(6):1693-704.
14. Kapadia, S. B. and Chisari, F. V. 2005 Hepatitis C virus RNA replication is regulated by host geranylgeranylation and fatty acids. Proc Natl Acad Sci USA. 102(7):2561-6.
15. Krepkiy, D. and Miziorko, H. M. 2004. Identification of active site residues in mevalonate diphosphate decarboxylase: implications for a family of phosphotransferases. Protein Sci. 13(7):1875-81.
16. Ku, E. C. 1996. Regulation of fatty acid biosynthesis by intermediates of the cholesterol biosynthetic pathway. Biochem Biophys Res Commun 225:173-9
17. Kuhn, R. J., Zhang, W., Rossmann, M. G., Pletnev, S. V., Corver, J., Lenches, E., Jones, C. T., Mukhopadhyay, S., Chipman, P. R., Strauss, E. G., Baker, T. S., and Strauss, J. H. 2002. Structure of dengue virus: implications for flavivirus organization, maturation, and fusion. Cell. 108(5):717-25
18. Ky B., Rader, D. J. 2005 The effects of statin therapy on plasma markers of inflammation in patients without vascular disease. Clin Cardiol. 28(2):67-70.
19. Lee, C. J., Lin, H. R., Liao, C. L., and Lin, Y. L. 2008. Cholesterol effectively blocks entry of flavivirus. J Virol 82, 6470-80.
20. Lindenbach, B. D. and Rice, C. M. 2003. Molecular biology of flaviviruses. Adv Virus Res. 59:23-61.
21. Mackenzie, J. M., Khromykh, A. A, and Parton, R. G. 2007. Cholesterol manipulation by West Nile virus perturbs the cellular immune response. Cell Host Microbe 2, 229-39.
22. Maksimova, E., Yie, T. A., and Rom, W. N. 2008. In vitro mechanisms of lovastatin on lung cancer cell lines as a potential chemopreventive agent. Lung. 186(1):45-54.
23. Medigeshi, G. R., Hirsch, A. J., Streblow, D. N., Nikolich-Zugich, J., and Nelson, J. A. 2008. West Nile virus entry requires cholesterol-rich membrane microdomains and is independent of alphavbeta3 integrin. J Virol 82, 5212-9.
24. Ng, C. Y., Gu, F., Phong, W. Y., Chen, Y. L., Lim, S. P., Davidson, A, and Vasudevan, S. G. 2007. Construction and characterization of a stable subgenomic dengue virus type 2 replicon system for antiviral compound and siRNA testing. Antiviral Res. 76(3):222-31.
25. Pokidysheva E, Zhang Y, Battisti A J, Bator-Kelly C M, Chipman P R, Xiao C, Gregorio G G, Hendrickson W A, Kuhn R J, Rossmann M G. 2006. Cryo-EM reconstruction of dengue virus in complex with the carbohydrate recognition domain of DC-SIGN. Cell. 124(3):485-93.
26. Pupo-Antúnez, M., Rodríguez, H., Vázquez, S., Vilaseca, J. C., Alvarez, M., Otero, A., and Guzmán, G. 1997. Monoclonal antibodies raised to the dengue-2 virus (Cuban: A15 strain) which recognize viral structural proteins. Hybridoma. 16(4):347-53.
27. Rigau-Perez, J. G., Clark, G. G., Gubler, D. J., Reiter, P., Sanders, E. J., and Vorndam A. V. 1998. Dengue and dengue haemorrhagic fever. Lancet 352:971-977.
28. Rothman, A. L. 2004. Dengue: defining protective versus pathologic immunity. J Clin Invest. 113: 946-951.
29. Srikiatkhachorn, A., Ajariyakhajorn, C., Endy, T. P., Kalayanarooj, S., Libraty, D. H., Green, S., Ennis, F. A., and Rothman, A. L. 2007 Virus-induced decline in soluble vascular endothelial growth receptor 2 is associated with plasma leakage in dengue hemorrhagic Fever. J Virol. 81(4):1592-600.
30. Vallett, S. M., Sanchez, H. B., Rosenfeld, J. M., and Osborne, T. F. 1996. A direct role for sterol regulatory element binding protein in activation of 3-hydroxy-3-methylglutaryl coenzyme A reductase gene. J Biol. Chem. 271(21):12247-53
31. Whitehead, S. S., Blaney, J. E., Durbin, A. P., and Murphy, B. R. 2007 BR.Prospects for a dengue virus vaccine. Nat Rev Microbial. 5(7):518-28.

What is claimed is:

1. A method of reducing Flavivirus viral replication in a subject, comprising administering to the subject a therapeutically effective amount of a mevalonate decarboxylase (MVD) antagonist, thereby reducing viral replication.

2. A method of reducing Flavivirus viral infection in a subject, comprising administering to the subject a therapeutically effective amount of a mevalonate decarboxylase (MVD) antagonist, thereby reducing viral infection.

3. The method of claim 1, wherein cholesterol synthesis is reduced.

4. The method of claim 1, wherein the production of isopentenyl 5-pyrophosphate from mevalonate 5-pyrophosphatate is reduced.

5. The method of any one of the preceding claims, wherein the Flavivirus virus is West Nile virus, Japanese encephalitis virus, or Dengue virus.

6. The method of claim 5, wherein the Flavivirus virus is Dengue virus.

7. The method of any one of the preceding claims, wherein the subject is human.

8. The method of any one of the preceding claims, wherein the antagonist is administered intravenously, intramuscularly, or subcutaneously to the subject.

9. The method of any of any one of the preceding claims, wherein the antagonist is administered in combination with a second therapeutic agent.

10. The method of any one of the preceding claims, wherein the antagonist is selected from the group consisting of an antibody, a small molecule, a nucleic acid, a fusion protein, and an MVD-derived peptidic compound.

11. The method of claim 10, wherein the small molecule is a statin, hymeglusin, or ZGA.

12. The method of claim 10, wherein the antibody is selected from the group consisting of a murine antibody, a human antibody, a humanized antibody, a bispecific antibody and a chimeric antibody.

13. The method of claim 10, wherein the antibody is selected from the group consisting of a Fab, Fab′2, ScFv, SMIP, affibody, avimer, nanobody, and a domain antibody.

14. The method of claim 10, wherein the nucleic acid is an antisense molecule selected from the group consisting of an RNA interfering agent and a ribozyme.
